(19) United States
(12) Patent Application Publication
(10) Pub. No.: US 2014/0171801 A1
(43) Pub. Date: Jun. 19, 2014

(54) MEDICAL DEVICE SUPPORT APPARATUS

(71) Applicant: GENERAL ELECTRIC COMPANY, Schenectady, NY (US)

(72) Inventors: Karen Starr, Monkton, MD (US); Lawrence Ten Eyck, Ellicott, MD (US); Thomas Jones, Columbia, MD (US)

(73) Assignee: GENERAL ELECTRIC COMPANY, Schenectady, NY (US)

(21) Appl. No.: 13/720,245

(22) Filed: Dec. 19, 2012

(51) Int. Cl. A61B 8/00

(52) U.S. Cl. CPC A61B 8/4218
USPC 600/459

(57) ABSTRACT

A support apparatus for a medical device is provided. The support apparatus comprises a hollow body having inner and outer surfaces, the inner surface defining a channel extending through the body along an axis, and at least two opposing wings coupled to the outer surface of the body and extending generally perpendicular to the axis, wherein channel is adapted to releasably receive the medical device and support the device in a fixed position.

### BACKGROUND OF THE INVENTION

The subject matter disclosed herein relates to a holder for a medical device such as an ultrasound probe.

Performing diagnostic ultrasonography requires manual dexterity of the probe. The probe is generally tethered to a cord which is secured to a distant device, such as a monitor or a computer base. The cord must remain flexible enough to allow for full rotation of the probe to enable full visualization, while also maintaining the sterile environment. Procedures often require the operator to manipulate both the probe, or other necessary sterile equipment, and the patient at the same time.

Conventional holders used in ultrasonography are often suspended over the patient. These holders not only may interfere with the operator's visual field, but they may also limit or block the operator's access to the patient field. Conventional holders also limit the flexibility of the probe and thereby inhibit rotation angle and/or manipulability of the probe.

### BRIEF DESCRIPTION OF THE INVENTION

The above-mentioned shortcomings, disadvantages and problems are addressed herein which will be understood by reading and understanding the following specification.

In an embodiment a support apparatus for a medical device is provided that includes a hollow body having inner and outer surfaces, the inner surface defining a channel extending through the body along an axis. The support apparatus further includes at least two opposing wings coupled to the outer surface of the body and extending generally perpendicular to the axis, wherein the channel is adapted to releasably receive the medical device and support the device in a fixed position.

In another embodiment, a support apparatus for an ultrasound probe is provided that includes a hollow body having inner and outer surfaces, the inner surface defining a channel and an axis therethrough. The support apparatus further includes a wing, integrally coupled to the outer surface of the body, and extending generally perpendicular to the axis, wherein the channel is adapted to releasably receive and support the ultrasound probe in a fixed position.

In another embodiment, an ultrasound probe support apparatus is provided that includes a hollow body that is generally dome shaped and comprises a flexible material, and wherein the body has an inner surface that defines a channel and an axis therethrough. The support apparatus further includes at least one wing coupled to the body, wherein the channel is adapted to receive and maintain the ultrasound probe in a fixed position.

Various other features, objects, and advantages of the invention will be made apparent to those skilled in the art from the accompanying drawings and detailed description thereof.

### BRIEF DESCRIPTION OF THE DRAWINGS

FIG. 1 is a cross sectional view of a holder in accordance with an embodiment;

FIG. 2 is an bottom view of a holder in accordance with an embodiment;

FIG. 3 is a top view of a holder in accordance with an alternate embodiment; and

FIG. 4 is a cross sectional view of a holder engaging an ultrasound probe in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

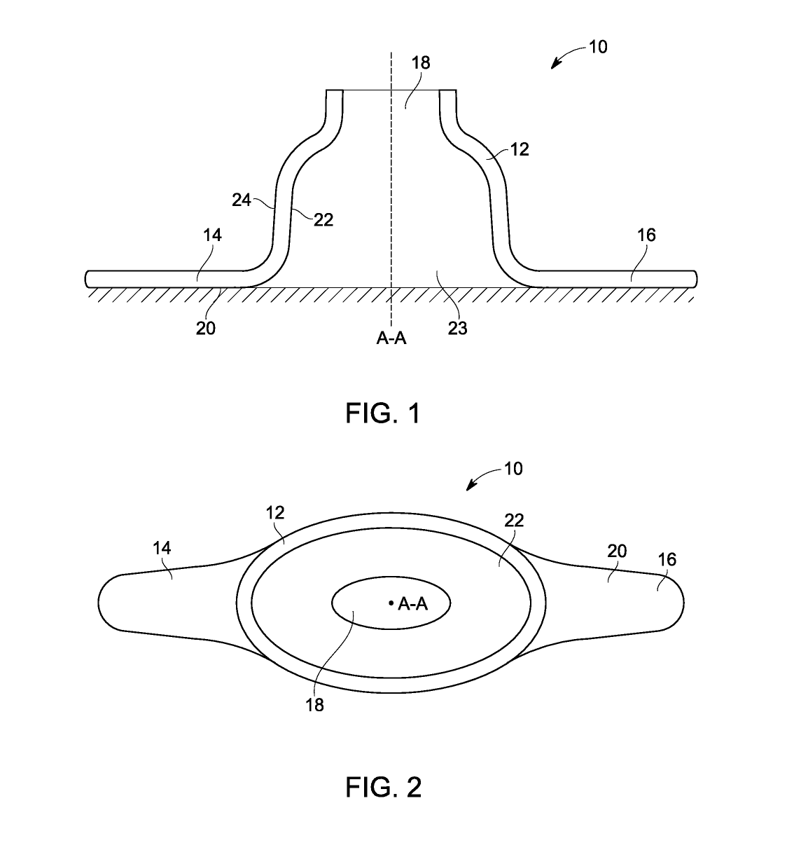

Referring to FIG. 1, a cross sectional view of a holder 10 is shown in accordance with an embodiment. The holder 10 will hereinafter be described as a holder for use with healthcare devices such as ultrasound probes. It should be appreciated, however, that other types of healthcare devices which are required to stay in contact or close proximity with the surface of a human or animal body may be envisioned for use with the holder 10.

In accordance with an embodiment, the holder 10 comprises a body 12 and wings 14, 16. The holder 10 is preferably generally elliptical in shape; however, other geometries are also envisioned. For example, the holder 10 may also be generally round or a quadrilateral to accommodate devices of various shapes. The holder 10 may be comprised of a lightweight and flexible material that is also durable and which preferably has a smooth and seamless outer surface for sterilization purposes. One such material is an elastomer such as silicone rubber that may be cast to the desired shape in a single piece using a mold. It should also be appreciated that the holder 10 may be disposable.

The body 12 is flexible, and generally dome shaped; however, other geometries are also envisioned. The body 12 defines a channel 18 and an axis A-A therethrough. The body 12 comprises a first (lower) surface 20, a second (inner) surface 22 and a third (outer) surface 24 (with the references to lower, inner and outer being relative to a supine patient). The first surface 20 is generally planar, but flexible in order to drape onto the contours of a patient (not shown).

Referring to FIG. 2, a bottom view of the holder 10 is shown in accordance with an embodiment. The first surface 20 is of a similar shape to that of the body 12. When the holder 10 is in use, the first surface 20 will be in contact with the patient forming a seal between the holder 10 and the patient. For purposes of this disclosure, the term seal is preferably defined to include any fastening or interface between two materials that prevents escape or entrance of a fluid, and the term fluid is preferably defined to include any substance that continually deforms under an applied shear stress and may therefore include both liquids and gases. It is preferable that the seal be hermetic or airtight, however, it should be appreciated that other types of seals are also envisioned. It should also be appreciated that the device may be rigid enough to maintain the probe in the desired position without first surface 20 forming a seal between the holder 10 and the patient.

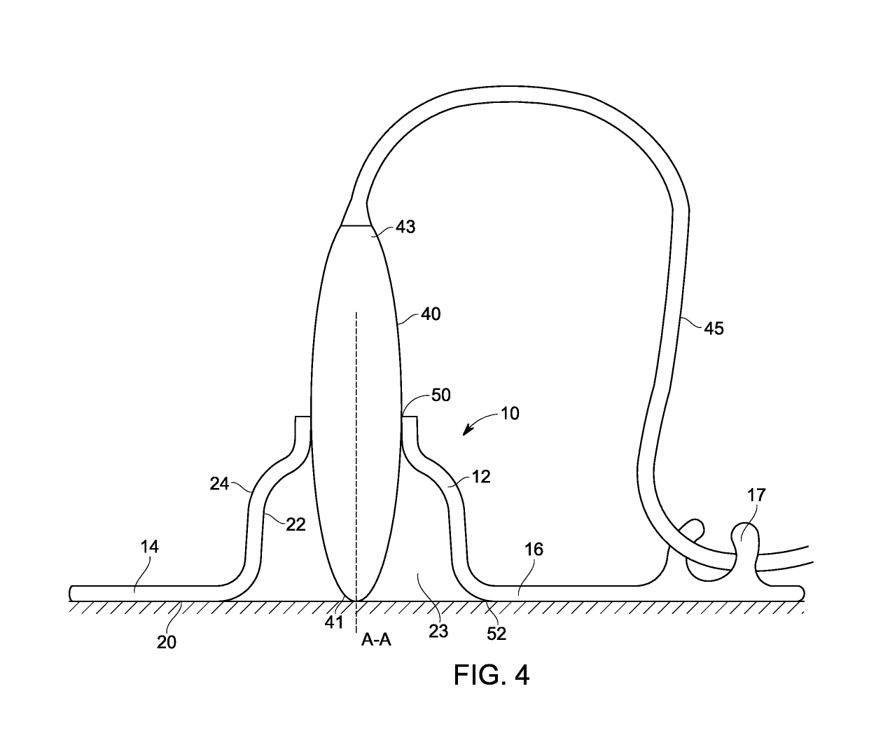

Referring back to FIG. 1, the second and third surfaces 22, 24 are generally domed in geometry. The second and third 22, 24 surfaces are preferably spaced between about 2 to 7 and millimeters apart, and most preferably about 3 millimeters apart. It should also be appreciated that the thickness between second and third 22, 24 surfaces may not be consistent within a single body 12, and instead may vary. For example, referring to FIG. 4, at edge 50 the second and third 22, 24 surfaces may be spaced 2 millimeters apart and the thickness increases to 4 millimeters at edge 52. The first and third surfaces 20, 24 may have similar or different dimensions. When the holder 10 is in use, the second surface 22 will not be in contact with the patient. The second surface 22 defines a hollow 23.

The wings 14, 16 extend outward from the body 12, generally perpendicular to axis A-A. In the depicted embodiment, the wings 14, 16 are integrally coupled to the body 12. However, it should be appreciated that the wings 14, 16 may also be detachably coupled to the body. This allows the operator to select wings of appropriate size or scale for the particular patient. Alternatively, the user may trim the wings 14, 16 with scissors, for example, to achieve the appropriate size or scale.

Also, in the depicted embodiment, the holder 10 comprises two wings 14, 16. The wings 14 and 16 are opposed and extend from the body 12 in order to stabilize the holder 10 when draped on a patient. However, it should also be appreciated that holder 10 may comprise more or fewer wings. Additionally, other arrangements of the wings 14, 16 are also envisioned. For example, a single wing 14 may extend uniformly about the circumference of body 12 for all 360 degrees. Alternatively, a plurality of visually distinct wings may extend about the circumference of body 12 at uniform or even irregular spacings.

The wings 14, 16 preferably comprise a self-adhering, elastomeric material, such as silicone rubber, in order for the wings 14, 16 to naturally attach and cling to the patient. However, it should be appreciated that the wings could comprise any other material that would be lightweight, flexible and durable and which provides a suitable level of sterilization.

In an alternate embodiment (not pictured), the wings 14, 16 may be comprised of a slightly rigid material and may be angularly or dome shaped in order to support the body 12 so that when the holder 10 is in use, the wings 14, 16 would not drape on the patient, but instead would extend over the patient and then contact the surface the patient is supported on, such as a patient table.

Referring now to FIG. 4, one or both of the wings 14, 16 may also comprise a clip 17. In the depicted embodiment, clip 17 is generally u-shaped and is adapted to releasably receive a power and/or communication cord 45 of an ultrasound probe 40. It should be appreciated, however, that other embodiments of clip 17 are envisioned. For example, the clip could comprise a flap that may be secured about the ultrasound cord 45 with a button, snap or other closure device. The clip 17 allows for the cord 45 to be draped in a way to help support the probe 40 in the desired position, and also aids in keeping the operator's field of view toward the patient free of the cord 45. It should also be appreciated that third surface 24 may also comprise a clip 17 (not pictured) in order to provide a path for the power and/or communication cord 45.

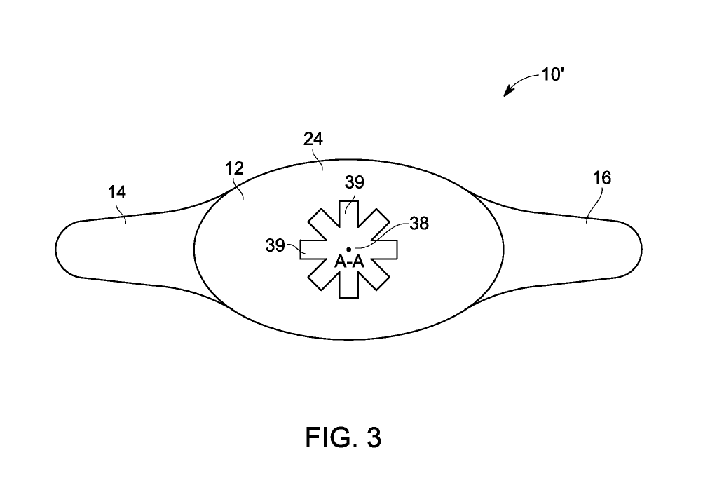

The channel 18 is sized and shaped to snuggly hold, for example, a medical device such as the probe 40. The channel 18 extends from a top or distal most (relative to a supine patient) edge 50 to a bottom or proximal most edge 52. The channel 18 is flexible, expandable and contractible. In the embodiment depicted in FIGS. 1 and 4, the channel is elliptical in geometry when viewed looking toward the patient along axis A-A. It should be appreciated however, that other cross-sectional geometries are envisioned to accommodate devices of various shapes. For example, referring to FIG. 3, a top view of a holder 10′ is shown in accordance with an alternate embodiment comprising a star-shaped channel 38 having grooves 39 arranged radially about axis A-A. Grooves 39 allow for the ultrasound probe 40 (or other medical device) to be inserted into channel 18 in various orientations with respect to axis A-A.

A method of using the holder 10 in connection with ultrasound scanning of a patient will now be described with reference to the embodiment of FIG. 4. In this illustrative embodiment, the holder 10 is coupled to the patient and supports the ultrasound probe 40. The method begins by an operator draping the holder 10 onto the patient so that the first surface 20 is in conforming contact with the patient's skin. Prior to or after draping the holder 10 onto the patient, the operator can apply ultrasound gel into hollow 23, which is capable of functioning as the ultrasound coupling gel reservoir. Once containing gel, the hollow 23 allows for the automatic application and replenishment of gel against the skin of the patient as the holder 10 is slid across the skin to a new location during a repositioning of the probe 40.

To place the ultrasound probe 40 in the holder 10, the operator pinches the outer surface 24 inwardly towards axis A-A and inserts the ultrasound probe 40 into the channel 18. When the operator releases the outer surface 24, the inner surface 22 contacts the probe 40, and the probe 40 is maintained snuggly within the channel 18 by friction and/or constriction. Additionally this may generate a mild vacuum further supporting the probe 40 in the selected position. The operator may then apply downward pressure along axis A-A so that the tip 41 of the ultrasound probe extends past the plane of the lower surface 20.

As the operator scans the patient, the operator may keep the probe 40 upright along axis A-A or, instead, a distal end 43 of the probe 40 may be tilted with respect to axis A-A. To change the angle of the probe 40 with respect to axis A-A, the operator may pinch the outer surface 24 inwardly towards axis A-A, thereby creating a gap between the channel 18 and an outer surface of the probe 40 at one or more locations about its circumference. The operator will then reposition the probe 40 to the desired angle with respect to axis A-A. Once the probe has been placed at the desired angle with respect to axis A-A, the operator will release the outer surface 24, whereby the inner surface 22 and channel 18 will again come into contact the probe 40. When the channel 18 contacts the probe 40 around a sufficient extent of its outer surface, the holder 10 will maintain the position of the probe 40. The operator may also pinch the outer surface 24 inwardly towards axis A-A in order to rotate the probe in channel 18 so as to orient the probe in an aligned way to a vessel or other organ structure or to obtain a cross sectional view.

The holder 10 described in accordance with the embodiment depicted in FIG. 1 provides several benefits over current probe holders used in ultrasonography. For example, the holder 10 can maintain the position of the probe 40 even when the operator is not touching the probe 40 or the holder 10, thereby supporting the probe 40 in an operational position to enhance clinical utility. Since the holder 10 rests upon the patient, it does not interfere with the operator's visual field, nor does it limit or block the operator's access to the patient field. Additionally, the hollow 23 defined by the lower surface 20 in conforming contact with the patient and the inner surface 22 of the holder 10, provides a convenient reservoir for the ultrasonic gel at a discrete area on the patient.

The holder 10 also improves the chances of a successful first procedure by freeing the users' hand or hands for other purposes during the intended ultrasound procedure thereby reducing errors and waste. Further, the holder 10 allows an inexperienced user to more easily develop the motor skills required to hold the probe while performing a procedure, thereby making the device easier to teach and sell.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. A support apparatus for a medical device, comprising:
a hollow body having inner and outer surfaces, the inner surface defining a channel extending through the body along an axis, and
at least two opposing wings coupled to the outer surface of the body and extending generally perpendicular to the axis,
wherein the channel is adapted to releasably receive the medical device and support the device in a fixed position.

2. The support apparatus of claim 1, wherein the medical device is an ultrasound probe.

3. The support apparatus of claim 1, wherein the wings are capable of supporting the body by extending over a supine patient or resting on a patient table.

4. The support apparatus of claim 1, wherein the wings are integrally coupled to the body.

5. The support apparatus of claim 4, wherein the body and wings comprise a flexible material.

6. The support apparatus of claim 5, wherein the body and wings comprise silicone rubber.

7. The support apparatus of claim 5, wherein at least one wing comprises an integrally formed clip adapted to releasably retain a medical device cord.

8. The support apparatus of claim 1, further including a lower surface capable of close confirming contact with a supine patient.

9. The support apparatus of claim 8, wherein the inner surface and the lower surface define a reservoir for receiving and maintaining an ultrasound gel.

10. A support apparatus for an ultrasound probe, comprising:
a hollow body having inner and outer surfaces, the inner surface defining a channel and an axis therethrough, and
a wing, integrally coupled to the outer surface of the body, and extending generally perpendicular to the axis,
wherein the channel is adapted to releasably receive and support the ultrasound probe in a fixed position.

11. The support apparatus of claim 10, wherein the body and wing comprise a flexible material.

12. The support apparatus of claim 11, wherein the body and wing comprise silicone rubber.

13. The support apparatus of claim 11, wherein the outer surface is capable of being pinched inwardly toward the axis in order to release the ultrasound probe to facilitate its repositioning from a first position to a second position.

14. The support apparatus of claim 11, wherein the outer surface is capable of being pinched inwardly toward the axis in order to release the ultrasound probe from the channel.

15. The support apparatus of claim 10, further including a lower surface capable of close confirming contact with a supine patient.

16. An ultrasound probe support apparatus comprising:
a hollow body that is generally dome shaped and comprises a flexible material, and wherein the body has an inner surface that defines a channel and an axis therethrough
at least one wing coupled to the body, and
wherein the channel is adapted to receive and maintain the ultrasound probe in a fixed position.

17. The ultrasound probe support apparatus of claim 16 wherein the inner surface of the body further defines a hollow that is adapted to constrain ultrasound gel.

18. The ultrasound probe support apparatus of claim 17, wherein the wing is integrally coupled to the outer surface of the body.

19. The ultrasound probe support apparatus of claim 18, wherein the wing comprises a clip adapted to releasably receive an ultrasound probe cord.

20. The ultrasound probe support apparatus of claim 17, wherein the outer surface is capable of being pinched inwardly toward the axis in order in order to release the ultrasound probe to facilitate its repositioning from a first position to a second position.

21. The ultrasound probe support apparatus of claim 20, wherein the outer surface is capable of being pinched inwardly toward the axis in order to release the ultrasound probe from the channel.

22. The ultrasound probe support apparatus of claim 21, wherein the body and wings comprise silicone rubber.
